(19) United States
(12) Patent Application Publication
(10) Pub. No.: US 2019/0382711 A1
(43) Pub. Date: Dec. 19, 2019

(54) HIGH-THROUGHPUT MICROFLUIDIC SCREENING IN DROPLETS

(71) Applicant: BIOMILLENIA SAS, Paris (FR)

(72) Inventors: Aleksander Dajkovic, Paris (FR); Dirk Löffert, Haan (DE)

(21) Appl. No.: 16/480,564

(22) PCT Filed: Jan. 26, 2018
(86) PCT No.: PCT/EP2018/051972

§ 371 Date: Jul. 24, 2019

(30) Foreign Application Priority Data
Jan. 26, 2017 (EP) 17153407.6
Aug. 28, 2017 (EP) 17188186.5

(51) Int. Cl. C12M 1/00; C12M 1/12; C12Q 1/04; C12N 1/02

(52) U.S. Cl. CPC C12M 47/04; C12N 1/02; C12Q 1/04; C12M 25/01

(57) ABSTRACT

In a first aspect, the present invention relates to a method for detecting and/or selecting a microorganism with a desired trait comprising the steps of (a) providing a composition comprising two or more microorganisms, (b) subjecting said two or more microorganisms to one of the following reactions leading to a change in the genetic composition in at least one microorganism, (i) natural transformation, (ii) transduction by phage, (iii) conjugation; (c) encapsulating each microorganism in a single microfluidic droplet together with an indicator molecule capable of detecting the presence of the desired trait, (d) incubating the encapsulated microorganism together with the indicator molecule under conditions that allow the detection of the desired trait, (e) sorting said droplets in a microfluidic system into at least (i) one group where the desired trait is not detectable and (ii) one group where the desired trait is detectable if present. In a second aspect, the present invention relates to a microorganism created by a method according to the first aspect of the present invention and its relative embodiments. In a third aspect, the present invention also encompasses a microfluidic kit comprising a microfluidic chip suitable for performing the method according to the first aspect and its relative embodiments and a collection reservoir for collecting single microfluidic droplets encapsulating the microorganism with the desired trait as defined in the second aspect and its relative embodiment. A variation of the method of the first aspect in which microorganisms not carrying a desired trait are brought in contact with nucleic acids carrying the trait is also disclosed.

### FIELD OF THE INVENTION

The invention is in the field of microorganisms strain development. It relates to the selection of microorganisms after transformation, transduction or conjugation using microfluidic systems, micro-cultures and droplets, preferably along with a fluorescent or colorimetric indicators. The present application is in the field of cell culture analysis. More precisely in the field of cell culture analysis on single cell level. The application is also in the field of microfluidics, particularly in the field of microfluidic analysis and devices.

### BACKGROUND

The production of biological compounds such as sugars, amino acids, antibiotics, carbon or nitrogen sources and other chemical building blocks is nowadays often efficiently performed in microorganisms. With the tools of genetic engineering it is possible to optimize microorganisms for an increased production of different compounds.

These optimized microorganisms are generated using mutagenic/combinatorial strategies capable to generate large libraries of genetically modified organisms. However, the drawback or bottleneck of all strategies is represented by the screening methods used to analyze individual library members.

The relevant screening methods are dependent on the molecules to be produced, but commonly the screening methods are based on chromatography and subsequent detection, in many cases by mass spectroscopy. A great disadvantage of the current methods is that parallelization and high throughput are difficult to achieve as the number of clones that can be analyzed is limited.

Frequently, preferences and the precautionary principle inhibit the development of bacterial strains for food applications and other market segments using recombinant DNA technology to produce genetically modified organisms (GMO's). According to the National Organic Program (NOP) of the U.S. Department of Agriculture's (USDA's) excluded methods are defined in the regulation as “a variety of methods used to genetically modify organisms or influence their growth and development by means that are not possible under natural conditions or processes, and are not considered compatible with organic production. Such methods include cell fusion, microencapsulation and macroencapsulation, and recombinant DNA technology (including gene deletion, gene doubling, introducing a foreign gene, and changing the positions of genes when achieved by recombinant DNA technology). Such methods do not include the use of traditional breeding, conjugation, fermentation, hybridization, in vitro fertilization, or tissue culture” (NOP § 205.2).

Non-GMO methods for strain development include transformation (the transfer of naked DNA between cells), transduction (the transfer of DNA carried by a virus) and conjugation or bacterial sex (direct transfer of DNA between cells in intimate contact with each other). These methods are powerful but limited by the ways in which transformed microorganisms can be isolated from a population. Classical ways include plating the microorganisms on selective culture media. This isolation methodology limits the creation of non-GMO strains that can be selected under such conditions.

Among the genetic tools for the creation and improvement of non-GMO strains of microorganisms for commercial and industrial applications, genetic transformation, transduction and conjugation carry a high potential to create new functionalities.

As of 2014 about 80 species of bacteria were known to be capable of transformation, about equally divided between Gram-positive and Gram-negative bacteria (Johnston et al., 2014 Nat. Rev. Microbiol. 12(3):181-196).

Among the industrially important lactic acid bacteria only Leuconostoc carnosum and Streptococcus thermophillus have demonstrated to be naturally competent to take up DNA and be transformed (Blomqvist et al., 2006 Appl. Environ. Microbiol. 72(10):6751-6756; Helmark et al., 2004 Appl. Environ. Microbiol. 70(6):3695-3699). The competence of S. themorphillus depends on the growth conditions and the growth medium and a competence stimulating peptide has been discovered (Gardan et al., 2009 J. Bacteriol. 191(14):4647-4655) and patented (US 2012/0040365 A1).

In bacterial conjugation, or bacterial sex, genetic material is transferred directly between cells in intimate physical contact through dedicated protruberances called pili (de la Cruz and Davies, 2000 Trends Microbiol. 8(3):128-133). Conjugation is widespread in bacteria and is routinely used in laboratories to transfer plasmids between relevant lactic acid bacteria. Important Lactococcus strains are improved by conjugation by the industry (Hpier et al., 2010 in Technology of Cheesemaking, Second Edition, Wiley-Blackwell, Oxford, UK).

Transduction is the transfer of genetic material between strains of bacteria mediated by bacterial viruses. Industrially important Lactococcus strains can be improved by transduction of plasmids between strains (McKay et al., 1973 J. Bacteriol. 115(3):810-815.) and plasmid transfer by transduction has even been demonstrated to occur from S. thermophillus to Lactococcus lactis (Ammann et al., 2008 J. Bacteriol. 190(8):3083-3087).

In the context of bacterial strains development, the power of traditional bacterial genetics to improve industrially and commercially important bacteria and create non-GMO strains is limited. Transformation, transduction and conjugation depend on the ability to select the few bacteria who have taken up and integrated the desired fragment of DNA from the vastly more abundant population of bacteria who haven't. This is traditionally done by plating for growth of desired transformed strain on selective culture media. The selective media employed either contain an antibiotic or lack an essential nutrient. This limits the possibility of transferring only the DNA containing either an antibiotic resistance gene, or DNA containing genetic material conferring prototrophy for a particular nutrient to an already auxotrophic strain. Antibiotic resistance genes are sometimes found in industrially important bacteria. Nevertheless, they are very rarely or almost never genetically linked with other desired genetic determinants thus rendering them useless for selection. Auxotrophy is widespread in industrially relevant bacteria and can be used as a selection marker (McKay et al., 1973 J. Bacteriol. 115(3):810-815) as long as the desired trait is genetically linked to the incoming genes with desired traits. At this stage, no known lactic acid bacterial strains improved by transduction are on the market (Johansen, Oregaard, Sorensen, Derkx, 2015 in Advances in Fermented Foods and Beverages, Woodhead Publishing).

In view of the above limitations affecting of the current methods, it is desirable to develop a different detection and/or selection methods to expand the arsenal of non-GMO development capabilities. The present invention responds to this need by providing the technology for selection of transformed microorganisms strains based on new activities and/or properties independent of their growth on culture media.

### SUMMARY OF THE INVENTION

In a first aspect, the present invention relates to a method for detecting and/or selecting a microorganism with a desired trait comprising the steps of (a) providing a composition comprising two or more microorganisms, (b) subjecting said two or more microorganisms to one of the following reactions leading to a change in the genetic composition in at least one microorganism, (i) natural transformation, (ii) transduction by phage, (iii) conjugation; (c) encapsulating each microorganism in a single microfluidic droplet together with an indicator molecule capable of detecting the presence of the desired trait, (d) incubating the encapsulated microorganism together with the indicator molecule under conditions that allow the detection of the desired trait, (e) sorting said droplets in a microfluidic system into at least (i) one group where the desired trait is not detectable and (ii) one group where the desired trait is detectable if present.

In a second aspect, the present invention relates to a microorganism created by a method according to the first aspect of the present invention and its relative embodiments.

In a third aspect, the present invention also encompasses a microfluidic kit comprising a microfluidic chip suitable for performing the method according to the first aspect and its relative embodiments and a collection reservoir for collecting single microfluidic droplets encapsulating the microorganism with the desired trait as defined in the second aspect and its relative embodiment.

### DESCRIPTION OF THE DRAWINGS

FIG. 1 shows a general scheme of the method according to the invention.

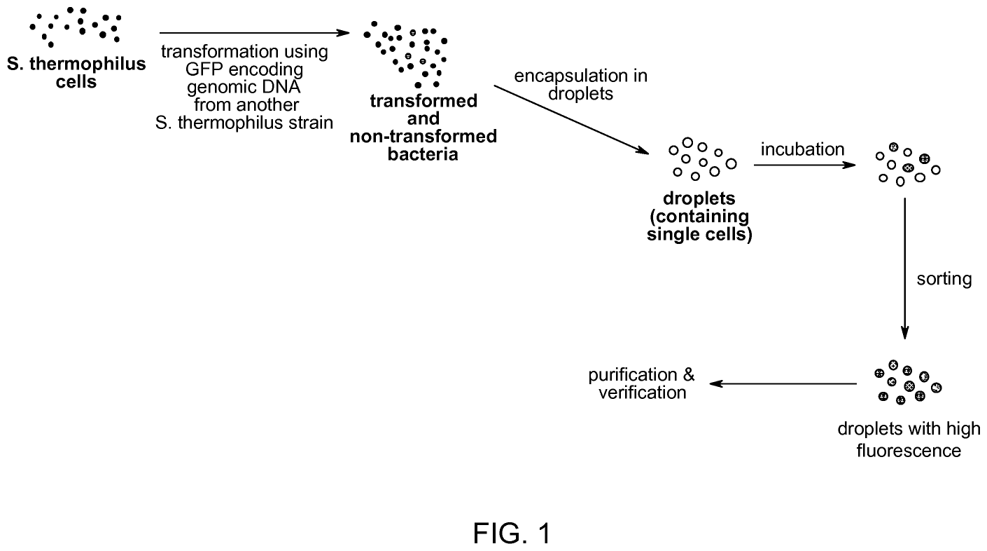

### DESCRIPTION OF THE INVENTION

The present invention is conceived to solve the limitations concerning the detection and/or selection of microorganisms that carry a desired trait after having undergone genetic transformation, transduction by phage, or conjugation, wherein the current methods are not practicable or inadequate. An additional advantage of the present invention over the current screening methods is represented by its high throughput in terms of automation in large scale and parallelization, providing the possibility to detect and/or select a plurality of transformed microorganisms in parallel.

The inventors have astonishingly found a way of rapidly detecting and/or selecting a naturally transformed microorganism that carry the desired trait by means of encapsulating each microorganism together with an indicator molecule in a single microfluidic droplet and detecting and/or selecting the transformed microorganism due to its interaction with said indicator molecule.

According to a first aspect, the present invention relates to a method for detecting and/or selecting a microorganism with a desired trait comprising the steps of (a) providing a composition comprising two or more microorganisms, (b) subjecting said two or more microorganisms to one of the following reactions leading to a change in the genetic composition in at least one microorganism, (i) natural transformation, (ii) transduction by phage, (iii) conjugation; (c) encapsulating each microorganism in a single microfluidic droplet together with an indicator molecule capable of detecting the presence of the desired trait, (d) incubating the encapsulated microorganism together with the indicator molecule under conditions that allow the detection of the desired trait, (e) sorting said droplets in a microfluidic system into at least (i) one group where the desired trait is not detectable and (ii) one group where the desired trait is detectable if present.

Genetic transformation is one of three processes for “horizontal gene transfer”, in which exogenous genetic material passes from a bacterium to another, the other two being conjugation (transfer of genetic material between two bacterial cells in direct contact) and transduction (injection of foreign DNA by a bacteriophage virus into the host bacterium). In transformation, the genetic material passes through the intervening medium and uptake is completely dependent on the recipient bacterium.

“Transformation” may also be used to describe the insertion of new genetic material into non-bacterial cells, including animal and plant cells; however, because “transformation” has a special meaning in relation to animal cells, indicating progression to a cancerous state, the process is usually called “transfection”.

Herein, the invention relates to “natural” transformation. This does not mean that the state of competence may not be actively induced. It means only that a natural system of transformation is used, i.e. not for example electroporation.

As used herein, the term “natural transformation” refers to a bacterial adaptation for DNA transfer that depends on the expression of numerous bacterial genes whose products appear to be responsible for this process. In general, transformation is a complex, energy-requiring developmental process. In order for a bacterium to bind, take up and recombine exogenous DNA into its chromosome, it must become competent, that is, enter a special physiological state. The DNA integrated into the host chromosome is usually (but with rare exceptions) derived from another bacterium of the same species and is thus homologous to the resident chromosome.

The capacity for natural transformation appears to occur in a number of prokaryotes and thus far 67 prokaryotic species (in seven different phyla) are known to undergo this process (Johnsborg et al., 2007 Res. Microbiol. 158(10):767-778).

As used herein, the term “microorganism” encompasses naked DNA or RNA, viruses, phages, bacteria, yeast or lysed bacteria and yeast.

In the context of the present invention, the term “desired trait” relates to a trait that is absent in the microorganism subjected to transformation. Therefore, the term “desired” may be used to define an “exogenous” trait. As used herein, the term “trait” refers to a phenotypic trait representing an observable and measurable characteristic generated by the expression of a determined set of genes. As used herein, the term “genetic composition” is referred to the genetic material characterizing the microorganism.

Essentially, there is a first and a second microorganism, wherein the first carries a trait that one would like to have in another microorganism, such as the second microorganism. It may happen that the first microorganism is merely naked DNA or a phage or a virus or a lysate of a microorganism. This is encompassed by the invention and the claims.

In one embodiment of the first aspect, at least one of the two or more microorganisms has the desired trait ab initio and at least one of the two or more microorganisms lacks said desired trait. As used herein, the term “ab initio” refers to the initial presence of the desired trait in the donor microorganism.

According to a preferred embodiment of the first aspect, the microorganisms are subjected to transformation.

In molecular biology, transformation is the genetic alteration of a cell resulting from the direct uptake and incorporation of exogenous genetic material from its surroundings through the cell membrane(s). For transformation to take place, the recipient bacteria must be in a state of competence, which might occur in nature as a time-limited response to environmental conditions such as starvation and cell density. However, since not all the bacterial species develop natural competence, said state of competence may also be “artificially” induced when cultured cells in laboratory are treated to make them transiently permeable to exogenous genetic material. Peptides are known to induce competence in some organisms.

According to another embodiment of the first aspect, the transformation may be actively induced. As used herein, the term “actively induced” is limited to the generation of the state of competence in the recipient microorganisms, e.g. bacteria, allowing the transformation to take place.

According to another embodiment of the first aspect, the transformation may be qualitatively and/or quantitatively determined. The execution of this embodiment is correlated to the method of detection adopted. Transformation is best quantitatively and qualitatively evaluated by the result of transformation, i.e. the appearance of transformants. The screening for the appearance of transformants is the one of the primary benefits of this invention.

In a further embodiment of the first aspect, suitable microorganisms, which might be genetically transformed to produce a compound include, but are not limited to bacterial strains, archaeal strains, fungal strains, yeast strains, algae, plant protoplasts, prokaryotic or eukaryotic cells, spores, insect cells or insect strains. In a preferred embodiment of the invention the microorganism which produces a compound of interest is a naked DNA or RNA, viruses, phages, bacterial strain, fungal strain and or yeast strain. In a most preferred embodiment the microorganism, which produces a compound, is a bacterial strain.

According to another embodiment of the first aspect, the microorganisms are bacteria and preferably the bacteria are not genetically modified organism (non-GMO). In the context of the present invention, the genus of bacteria is selected from the group comprising Carnobacterium, Enterococcus, Lactobacillus, Lactococcus, Lactosphaera, Leuconostoc, Melissococcus, Oenococcus, Pediococcus, Streptococcus, Tetragenococcus, Vagococcus and Weissella.

In an alternative embodiment, the microorganisms are fungi and preferably the fungi are not genetically modified organism (non-GMO). In the context of the present invention, the genus of fungi is selected form the group comprising Aspergillus, Saccharomyces, Rhizopus, Neurospora, Cephalosporium and Penicillium.

The trait may be encoded on a plasmid or on the chromosome of the first microorganism. The first and the second microorganism are incubated under conditions that allow the transfer of the DNA or RNA from one microorganism (the first) to the other (the second).

Incubation may be performed in any way possible. Therefore, droplets may be incubated outside of a microfluidic device and later be subjected to a microfluidic device. Alternatively, the incubation may take place within the microfluidic system. However, it is important that the microfluidic droplet remains intact during the incubation.

Incubation time has to be selected accordingly. In general, the incubation time needs to be long enough to allow for the microorganisms to grow and produce and desired trait.

After incubation, the droplets are analyzed in a microfluidic device, screening for the presence of the desired trait. The detection method is dependent on the indicator molecule used. Therefore, if the indicator molecule in detecting the desired trait generates a fluorescent signal, the detection method is fluorescence detection.

The microfluidic droplets comprising the microorganisms may be additionally encapsulated to separate the contents from the environment. A possible method of encapsulation is discussed in WO 2010/063937 A1. The microfluidic droplets are separated from the environment using a phase immiscible with the medium to separate or encapsulate droplets. The immiscible phase may be an oil, e.g. a fluorinated oil.

According to another embodiment of the first aspect, the microfluidic droplet comprises two immiscible phases, wherein the microorganism is in the aqueous phase.

Each microorganism is encapsulated into a single microfluidic droplet together with an indicator molecule and subjected to incubation into a microfluidic system.

Preferably, the microfluidic droplet comprises two immiscible phases and wherein the microorganism is in the aqueous phase. The indicator molecule is included in the aqueous phase, or subsequently injected.

In another embodiment of the first aspect, the indicator molecule is selected from the group comprising a detector molecule, enzyme, antibody, label, fluorescent dye, colorimetric dye, viscosity sensor, ion detection sensor and pH.

Detection may also be performed with a reporter gene. In the context of the present invention an activated reporter gene or the activity of the reporter gene refers to the expression of a detectable gene product. Said gene product might be continuously expressed or the expression might be triggered under certain conditions.

According to a preferred embodiment of the first aspect, the expression of the desired trait determines the production of a compound selected from the group comprising saccharide, protein, amino acid, enzyme, lipid and polysaccharide. While preferred produced compounds are described hereinafter, the expression of the desired trait may also modify the behavior of the transformed microorganism by improving or diminishing the resistance to a chemical, the adaptation to a culture media and/or condition and, an increased or decreased rate of growth under desired conditions.

According to another embodiment of the first aspect, the desired trait may be expressed on the surface of the transformed microorganism, it may be intracellular, secreted and released extracellularly. Therefore, the produced compound of interest might be any compound, which can be exported or secreted into the droplet medium by the microorganism and which can be detected by an indicator molecule.

In another embodiment of the first aspect, the detection may be performed optically, by means of pH, electrically, or by means of change in viscosity. In a preferred embodiment, the reporter gene for detection encodes a fluorescent protein such as green fluorescent protein (GFP), a variant of GFP, yellow fluorescent protein (YFP), a variant of YFP, red fluorescent protein (RFP), a variant of RFP, cyan fluorescent protein (CFP), a variant of CFP or the reporter gene operon is a luminescence operon such as the lux operon. It is known to the person skilled in the art that homologs of said proteins may be used.

The method disclosed herein may find different applications including the development of non-genetically modified (GM) microorganisms. This is encompassed by a second aspect of the present invention.

In the context of the present invention, the term “created” relates to the change in the genetic composition of at least one microorganism, i.e. the recipient microorganism. Said change may allow the recipient microorganism to produce a desired phenotype or alter the expression of an endogenous trait. As used herein, the term “created” is not limited to indicate a microorganism subjected to a natural transformation reaction, but it may refer to transduction by phage and conjugation. wherein exogenous genetic material is transmitted from a donor microorganism to a recipient microorganism.

In an embodiment of the second aspect, the microorganism created by the method of the first aspect is suitable for industrial and/or commercial use.

The expressed exogenous trait may have either a direct commercial value or may serve as an intermediate in the production of a subsequent compound, which has commercial value. The produced compound may also have an industrial value that finds application in drug production, food processing, bio-control agents, enzyme biotechnology as well as in research and development.

In the context of the present invention, preferred produced compounds having an industrial and/or commercial use include, but are not limited to primary metabolites: L- and D- amino acids, sugars and carbon sources such as L-arabinose, N-acetyl-D-glucosamine, N-acetyl-D-mannosamine, N-acetylneuraminate, lactose, L- and D-lactate, D-glucosamine, D-glucose-6-phosphate, D-xylose, D-galactose, glycerol, maltose, maltotriose and melibiose; nucleosides such as cytidine, guanine, adenine, thymidine, guanosine, adenosine; lipids such as hexadecanoate and glycerol 3-phosphate; indole, maltohexose, maltopentose, putrescine, spermidine, ornithine, tetradecanoate and nicotinamide adenine dinucleotide, as well as volatile compounds such as diacetyl, acetoin, 1,2-butanediol, isovaleric acid, etc.

Further relevant compounds of interest include, but are not limited to, secondary metabolites. Such metabolites can be produced naturally by the transformed microorganism but may also be generated via a heterologous biosynthetic pathway introduced into the microorganisms by genetic engineering. Examples of secondary metabolites include, but are not limited to, polyketides (such as erythryomycin and avermectins), small molecules (such as resveratrol, steviol and artemisenin) or non-ribosomal peptides.

According to a third aspect, the present invention also encompasses a microfluidic kit comprising a chip suitable for performing the method according to the first aspect and its relative embodiments and a collection reservoir for collecting single microfluidic droplets encapsulating the microorganism with the desired trait as defined in the second aspect and its relative embodiment.

As outlined above, the invention attempts to transfer desired genetic information from one organism known to have this trait to another organism not known to have this trait. The inventors facilitate the detection of such a transfer by means of their microfluidic detection system. This, however may also be done in alternative way. Here, there is only the target organism present. The trait comes from free nucleic acids in the composition.

The invention also relates to a method for detecting and/or selecting a microorganism with a desired trait comprising the steps of
a. providing a composition comprising
   i. one or more microorganisms not carrying the desired trait and,
   ii. one or more nucleic acid molecules encoding the desired trait,
b. subjecting said two or more microorganisms to one of the following reactions leading to a change in the genetic composition of the at least one microorganism,
   i. natural genetic transformation
   ii. transduction by phage
   iii. conjugation
c. encapsulating each microorganism in a single microfluidic droplet preferably together with an indicator molecule capable of detecting the presence of the desired trait,
d. incubating the encapsulated microorganism together with the indicator molecule under conditions that allow the detection of the desired trait,
e. or detecting the desired trait based on a change of the phenotype of the organism in the absence of an indicator molecule,
f. sorting said droplets in a microfluidic system into at least
   i. one group where the desired trait is not detectable and
   ii. one group where the desired trait is detectable.

A further alternative of the invention lies in the use of no indicator molecule. Here, the desired trait is detected by a change of the phenotype of the organism.

Preferably, the invention also relates to a method for detecting and/or selecting a microorganism with a desired trait comprising the steps of
a. providing a composition comprising
   iii. one or more microorganisms not carrying the desired trait and,
   iv. one or more nucleic acid molecules encoding the desired trait,
b. subjecting said two or more microorganisms to one of the following reactions leading to a change in the genetic composition of the at least one microorganism,
   v. natural genetic transformation
   vi. transduction by phage
   vii. conjugation
c. detecting the desired trait based on a change of the phenotype of the.

The step d.) sorting said droplets in a microfluidic system into at least, one group where the desired trait is not detectable and one group where the desired trait is detectable may be optionally added.

Finally, the inventions relates to microorganisms obtained or obtainable by one of the methods according to the invention.

1. Method for detecting and/or selecting a microorganism with a desired trait comprising the steps of
a. providing a composition comprising two or more microorganisms,
b. subjecting said two or more microorganisms to one of the following reactions leading to a change in the genetic composition in at least one microorganism,
i. natural genetic transformation
ii. transduction by phage
iii. conjugation
c. encapsulating each microorganism in a single microfluidic droplet together with an indicator molecule capable of detecting the presence of the desired trait,
d. incubating the encapsulated microorganism together with the indicator molecule under conditions that allow the detection of the desired trait,
e. sorting said droplets in a microfluidic system into at least
i. one group where the desired trait is not detectable and
ii. one group where the desired trait is detectable.

2. The method according to claim 1, wherein at least one of said two or more microorganisms has the desired trait ab initio and at least one of said two or more microorganisms lacks said desired trait.

3. The method according to any of the claims 1 and 2, wherein at least one microorganism is subjected to natural genetic transformation.

4. The method according to any of the claims 1 to 3, wherein the natural genetic transformation is actively induced,

5. The method according to any of the claims 1 to 4, wherein the transformation is qualitatively and/or quantitatively determined.

6. The method according to any of the claims 1 to 5, wherein the microorganism is selected from the group comprising naked DNA or RNA, viruses, phages, bacterial strain, fungal strain, yeast strain, and lysate of bacterial strain, fungal strain or yeast strain.

7. The method according to any of the claims 1 to 6, wherein the microorganisms are bacteria and preferably the bacteria are non-genetically modified organisms (non-GMO).

8. The method according to any of the claims 1 to 7, wherein the microfluidic droplet comprises two immiscible phases and wherein the microorganism is in the aqueous phase.

9. The method according to any of the claims 1 to 8, wherein the indicator molecule is selected from the group comprising a detector molecule, enzyme, antibody, label, fluorescent dye, colorimetric dye, viscosity sensor, ion detection sensor and pH sensor.

10. The method according to any of the claims 1 to 9, wherein the desired trait is selected from the group comprising the production of a saccharide, protein, amino acid, enzyme, lipid, polysaccharide, the resistance to a chemical, the adaptation to a culture media and/or condition and an increased or decreased rate of growth under a desired condition.

11. The method according to any of the claims 1 to 10, wherein the desired trait is expressed on the surface of the transformed microorganism, intracellularly expressed, or secreted into the medium.

12. The method according to any of the claims 1 to 11, wherein the detection is performed optically, by means of pH, electrically or by means of change in viscosity or preferably by fluorescence.

13. Microorganism obtained or obtainable by a method according to any of the claims 1 to 12.

14. A microfluidic kit, the kit comprising:
a microfluidic chip suitable for performing the method according to any of the claims 1 to 12;
a collection reservoir for collecting single microfluidic droplets encapsulating the microorganism according to any of the claims 13 and 14 and optionally,
a competent cell.

15. Method for detecting and/or selecting a microorganism with a desired trait comprising the steps of
a. providing a composition comprising
i. one or more microorganisms not carrying the desired trait and,
ii. one or more nucleic acid molecules encoding the desired trait,
b. subjecting said two or more microorganisms to one of the following reactions leading to a change in the genetic composition of the at least one microorganism,
i. natural genetic transformation
ii. transduction by phage
iii. conjugation
c. encapsulating each microorganism in a single microfluidic droplet preferably together with an indicator molecule capable of detecting the presence of the desired trait,
d. incubating the encapsulated microorganism together with the indicator molecule under conditions that allow the detection of the desired trait,
e. or detecting the desired trait based on a change of the phenotype of the organism in the absence of an indicator molecule,
f. sorting said droplets in a microfluidic system into at least
i. one group where the desired trait is not detectable and
ii. one group where the desired trait is detectable.

16. Microorganism obtained or obtainable by a method according to claim 15.
